(19) United States
(12) Patent Application Publication
(10) Pub. No.: US 2022/0008243 A1
(43) Pub. Date: Jan. 13, 2022

(54) SYSTEMS AND DEVICES FOR MONITORING AND TREATING BRUXISM

(71) Applicant: BRUXA, INC., Los Angeles, CA (US)

(72) Inventors: Pablo OSORIO MARTINI, Los Angeles, CA (US); Joseph UPCHURCH, Culver City, CA (US)

(21) Appl. No.: 17/292,383

(22) PCT Filed: Nov. 7, 2019
(86) PCT No.: PCT/US19/60331

§ 371 Date: May 7, 2021

Related U.S. Application Data
(60) Provisional application No. 62/814,723, filed on Mar. 6, 2019.
(60) Provisional application No. 62/757,743, filed on Nov. 8, 2018.

(51) Int. Cl. A61F 5/56; A61B 5/00; A61B 5/01; A61B 5/11; A61L 2/10

(52) U.S. Cl. CPC A61F 5/566; A61B 5/0008; A61B 5/01; A61B 5/11; A61B 5/486; A61L 2202/122; A61L 2/10; A61F 2005/563; A61B 2562/0271; A61B 2560/0214; A61B 2562/0219; A61B 5/682

(57) ABSTRACT

Systems, apparatuses, software, and methods for monitoring and/or treating a health condition such as bruxism. The devices and apparatuses described herein can include a smart night guard, a smart case, and an electronic device. The smart night guard comprises at least one pressure sensor and a transmitter to send sensor data to the smart case or electronic device. The sensor data can be analyzed to provide an evaluation of the health condition.

### CROSS-REFERENCE

This application claims the benefit of U.S. Provisional Application No. 62/757,743, filed Nov. 8, 2018, and U.S. Provisional Application No. 62/814,723, filed Mar. 6, 2019, each of which is hereby incorporated herein by reference.

### BACKGROUND

Bruxism is a problem that is characterized by the unconscious grinding and clenching of teeth. The etiology of bruxism is unclear, but one of the attributed causes include stress. Consequences of bruxism include tooth wear, signs and symptoms of temporomandibular disorders (TMD), headaches, toothache, mobile teeth, interrupted sleep, and various problems with dental restorations as well as with fixed and removable prostheses.

### SUMMARY

The present disclosure includes systems, apparatuses, software, and methods for monitoring and/or treating oral-related disorders or conditions such as bruxism or teeth grinding. The present disclosure addresses various problems with existing tools for dealing with bruxism. For example, the most common tool that dentists use for treating patients that grind or clench their teeth is a device known as a night guard or mouth guard that is customized to fit a patient's upper or lower teeth. However, the only function of this device is teeth protection, which offers no ability to track, reduce, diagnose, or cure bruxism episodes. Dentists have relied on polysomnography and various versions of electromyography (EMG) to study bruxism. Other inventions have tried to approach the problem of diagnosing and treating bruxism through devices including a mouth guard and pressure sensors which are electrically coupled with biofeedback mechanisms such as vibration, sounds or electrical stimulation to indicate that bruxism is occurring. However, the present disclosure recognizes that those alarm mechanics can cause sleep disruption and fail to solve the problem through identification of the root causes of bruxism. Since one of the root causes of bruxism can be attributed to stress, there are multiple recommended treatments to reduce bruxism that are based on behavioral approaches. Thus, there is a need for a device that not only protects the teeth and provides data of bruxism episodes, but also treats bruxism through biofeedback by providing information that could trigger behavioral changes on people. Additionally, the present disclosure addresses a need for a device that can be used to better measure compliance in wearing the device than current devices that measure compliance based on time near body temperature, which can easily be deceived.

In some aspects, described herein is a system for monitoring and/or treatment of bruxism. In some embodiments, the system obtains user data to determine correlations or associations between teeth grinding/clenching and other activities. In some embodiments, the user data comprises sensor data from an electronic night guard indicative of the duration and/or intensity of teeth grinding and/or clenching. In some embodiments, the user data comprises user activities such as behavioral activities or therapies. These behavioral activities may be for the purpose of treating bruxism (e.g., reducing duration and/or intensity of teeth grinding/clenching) or alleviating symptoms of bruxism (e.g., headaches, poor sleep quality).

In some embodiments, an electronic night guard (“smart night guard”) comprises one or more sensors for detecting teeth grinding and/or clenching. In some embodiments, the electronic night guard comprises a wireless transmitter for communicating with another electronic device. In some embodiments, the electronic night guard transmits data to the electronic device in real-time. Alternatively or in combination, data is transmitted periodically or at discrete time points. In some cases, the data is transmitted based on whether the electronic device being within communication range, there is sufficient power, or other relevant metrics.

In some embodiments, user data is analyzed to generate an evaluation of the user's disorder or condition. In some embodiments, the sensor data is analyzed at least in part by correlating user's data on teeth grinding and/or clenching with user's behavioral activities. In some embodiments, the system includes a smart night guard device with electronic components embedded into a mouthpiece that is customized to the user's mouth. In some embodiments, the smart night guard measures and records teeth grinding and clenching through an embedded pressure sensor and optionally additional data such as any of respiratory rate, heart rate, and movement through an embedded accelerometer. In some embodiments, the system comprises a smart case configured to charge the smart night guard and optionally stores at least one of pressure, motion, or temperature data collected through the smart night guard. In some embodiments, the smart case is configured to transfer the stored data to a smart device such as, for example, a smartphone, tablet, or personal computer. In some embodiments, the smart device is configured with the ability to record and/or store behavioral activities. In some embodiments, the behavioral activities are entered by the user and/or automatically detected (e.g., via sensor data). In some embodiments, the user enters behavioral activities as inputs in a software application on the smart device (e.g., a “bruxism application”). In some embodiments, the software application is configured to process the data output from the smart night guard and/or other user inputs to provide analysis such as, for example, statistical correlations of the data to show the effectiveness of recommended treatments for bruxism that are based on behavioral changes. In some embodiments, the data is displayed or sent to the user for purposes of triggering a change in behavior (or continuing with a given behavior such as a particular treatment). In some embodiments, the analysis is presented to the user to encourage the user to adopt, change, or continue behavior or behavioral activities for purposes of reducing the frequency, force and/or duration of bruxism episodes and/or improving sleep quality.

In some aspects, disclosed herein is a system for monitoring a health condition, comprising: (a) a smart night guard comprising: (i) an inner layer configured to detachably engage at least one tooth of a user; (ii) at least one pressure sensor configured to generate sensor data in response to detection of an application of pressure to the inner layer; and (iii) a transmitter in communication with the at least one pressure sensor, said transmitter configured to transmit the sensor data obtained from the at least one pressure sensor; (b) a smart case comprising a processor and non-transitory computer readable medium including instructions executable by the processor and configured to cause the processor to: (i) receive the sensor data transmitted by the smart night guard over a period of time; (ii) store the sensor data in a local memory; and (iii) send the sensor data to an electronic device; (c) the electronic device comprising a processor and non-transitory computer readable medium including instructions executable by the processor and configured to cause the processor to: (i) receive the sensor data transmitted by the smart case over a time period; (ii) obtain user data comprising at least one user activity that is temporally proximate to the time period; and (iii) analyze the sensor data and the user data, thereby generating an output indicative of the health condition. In some embodiments, the smart night guard further comprises at least one sensor. In some embodiments, the at least one sensor comprises a motion sensor, a temperature sensor, a pH sensor, a gyroscope, a geomagnetic sensor, or any combination thereof. In some embodiments, the sensor data comprises absolute orientation, angular velocity vectors, acceleration vectors, magnetic field strength vectors, linear acceleration vectors, gravity vectors, ambient temperature, or any combination thereof. In some embodiments, the smart night guard is configured to increase sampling rate of the at least one pressure sensor when the pressure exceeds a minimum threshold or a moving threshold. In some embodiments, the smart night guard comprises an anti-microbial coating. In some embodiments, the smart night guard is configured to transmit the sensor data when the pressure exceeds a minimum threshold or a moving threshold. In some embodiments, the smart night guard is configured to temporarily store the sensor data and transmit the sensor data when charging, upon receiving a signal from the smart night guard or electronic device, when the smart night guard is experiencing minimal motion, or any combination thereof. In some embodiments, the smart night guard is configured to detect improper device states indicative of a defect or malfunction. In some embodiments, at least one of the smart night guard, smart case, or electronic device provides a signal or message to discontinue use upon detection of the defect or malfunction. In some embodiments, the signal or message to discontinue use comprises a light, a sound, or written message or warning. In some embodiments, the smart night guard is configured to calibrate the sensor data based on pressure corresponding to maximum or minimum detected bite force. In some embodiments, the sensor data comprises intensity and duration of the pressure. In some embodiments, the smart case comprises a support for holding the smart night guard. In some embodiments, the smart case comprises a wireless charger for charging the smart night guard. In some embodiments, the wireless charger comprises a first induction coil, and the smart night guard comprises a second induction coil, wherein the smart night guard undergoes inductive charging when positioned so the first induction coil and the second induction coil are in proximity. In some embodiments, at least one of the smart case or the smart night guard comprises at least one magnet for positioning the smart night guard within the smart case to allow inductive charging. In some embodiments, the smart case comprises at least one magnet for positioning the smart night guard. In some embodiments, the smart case comprises a display for providing information to the user. In some embodiments, the smart case comprises a UV light source for disinfecting the smart night guard. In some embodiments, the smart case comprises an anti-microbial coating. In some embodiments, the output comprises an evaluation of the health condition of the user. In some embodiments, the health condition comprises an oral health condition or a sleeping disorder or condition. In some embodiments, the health condition comprises bruxism, sleep apnea, insomnia, snoring, restless leg syndrome, sleepwalking. In some embodiments, the output comprises a correlation between the at least one user activity and an intensity or severity of the health condition of the user. In some embodiments, the output comprises a relationship between the sensor data and the at least one behavioral activity. In some embodiments, the output comprises a risk prediction of a health condition of the user. In some embodiments, the risk prediction is generated by a machine learning classifier configured to assess the risk of the health condition based on the sensor data. In some embodiments, the risk prediction comprises a degree of risk of the health condition. In some embodiments, the output comprises a number of bruxism episodes, maximum and average force of each bruxism episode, maximum and average durations of the bruxism episodes, an amount or quality of sleep, compliance to wearing the device, or any combination thereof. In some embodiments, the output comprises a score generated based on number of bruxism episodes, maximum and average force of each bruxism episode, maximum and average durations of the bruxism episodes, an amount or quality of sleep, compliance to wearing the device, or any combination thereof. In some embodiments, the output comprises at least one of personal or community correlation between the health condition and behavioral activity or therapeutic treatment. In some embodiments, behavioral activity or therapeutic treatment comprises dietary activity, physical activity, personal well-being indicator, or any combination thereof. In some embodiments, dietary activity comprises consumption of caffeine, alcohol, fats, simple carbohydrates, complex carbohydrates, protein, vitamins, supplements, or any combination thereof. In some embodiments, physical activity comprises aerobic exercise, anaerobic exercise, stretching, meditation, yoga, or any combination thereof. In some embodiments, personal well-being indicator comprises self-reported stress level, energy level, mental state, or any combination thereof. In some embodiments, the electronic device is further configured to generate a recommendation based on the output. In some embodiments, the recommendation comprises modifying the at least one behavioral activity, beginning a new behavioral activity, or terminating the at least one behavioral activity. In some embodiments, the recommendation comprises at least one therapeutic treatment or activity for reducing at least one of frequency, force, of duration of bruxism episodes. In some embodiments, the recommendation comprises at least one therapeutic treatment or activity for improving sleep quality. In some embodiments, the recommendation comprises at least one therapeutic treatment or activity for improving a symptom of the health condition. In some embodiments, the electronic device is configured to determine user compliance to the recommendation. In some embodiments, user compliance is determined at least partly based on user input. In some embodiments, the system is configured to monitor the health condition over time by continuously or repeatedly collecting sensor data and user data comprising the at least one behavioral activity. In some embodiments, the system is configured to generate updated output over time. In some embodiments, the system is adapted for continuous, repeated, or periodic monitoring of the user for at least 1 week, 2 weeks, 3 weeks, 4 weeks, 5 weeks, 6 weeks, 7 weeks, 8 weeks, 9 weeks, 10 weeks, 11 weeks, or at least 12 weeks. In some embodiments, the smart night guard is not configured to provide a biofeedback mechanism for the health condition, the biofeedback mechanism comprising vibration, sounds, or electrical stimulation. In some embodiments, the at least one pressure sensor comprises a single circuit having an arch shape configured to allow detection of the pressure from any point of contact between upper and lower teeth. In some embodiments, the electronic device is configured integrate the sensor data and output with a third party application. In some embodiments, the electronic device is configured integrate the sensor data and output with data from a third party application or sensor. In some embodiments, the electronic device is configured to generate a report comprising historical sensor data, user data, and outputs. In some embodiments, the electronic device is configured to provide a user portal comprising historical sensor data, user data, outputs, smart night guard status, or any combination thereof.

In some aspects, disclosed herein is a system for monitoring a health condition, comprising: (a) a smart night guard comprising: (i) an inner layer configured to detachably engage at least one tooth of a user; (ii) at least one pressure sensor configured to generate sensor data in response to detection of an application of pressure to the inner layer; and (iii) a transmitter in communication with the at least one pressure sensor, said transmitter configured to transmit the sensor data obtained from the at least one pressure sensor; (b) a smart case comprising a processor and non-transitory computer readable medium including instructions executable by the processor and configured to cause the processor to: (i) receive the sensor data transmitted by the smart night guard over a period of time; (ii) store the sensor data in a local memory; and (iii) send the sensor data to an electronic device. 54. A system for monitoring a health condition, comprising: (a) a smart night guard comprising: (i) an inner layer configured to detachably engage at least one tooth of a user; (ii) at least one pressure sensor configured to generate sensor data in response to detection of an application of pressure to the inner layer; and (iii) a transmitter in communication with the at least one pressure sensor, said transmitter configured to transmit the sensor data obtained from the at least one pressure sensor; (b) the electronic device comprising a processor and non-transitory computer readable medium including instructions executable by the processor and configured to cause the processor to: (i) receive the sensor data transmitted by the smart night guard over a time period; (ii) obtain user data comprising at least one user activity that is temporally proximate to the time period; and (iii) analyze the sensor data and the user data, thereby generating an output indicative of the health condition. In some embodiments, the smart night guard further comprises at least one sensor. In some embodiments, the at least one sensor comprises a motion sensor, a temperature sensor, a pH sensor, a gyroscope, a geomagnetic sensor, or any combination thereof. In some embodiments, the sensor data comprises absolute orientation, angular velocity vectors, acceleration vectors, magnetic field strength vectors, linear acceleration vectors, gravity vectors, ambient temperature, or any combination thereof. In some embodiments, the smart night guard is configured to increase sampling rate of the at least one pressure sensor when the pressure exceeds a minimum threshold or a moving threshold. In some embodiments, the smart night guard comprises an anti-microbial coating. In some embodiments, the smart night guard is configured to transmit the sensor data when the pressure exceeds a minimum threshold or a moving threshold. In some embodiments, the smart night guard is configured to temporarily store the sensor data and transmit the sensor data when charging, upon receiving a signal from the smart night guard or electronic device, when the smart night guard is experiencing minimal motion, or any combination thereof. In some embodiments, the smart night guard is configured to detect improper device states indicative of a defect or malfunction. In some embodiments, at least one of the smart night guard, smart case, or electronic device provides a signal or message to discontinue use upon detection of the defect or malfunction. In some embodiments, the signal or message to discontinue use comprises a light, a sound, or written message or warning. In some embodiments, the smart night guard is configured to calibrate the sensor data based on pressure corresponding to maximum or minimum detected bite force. In some embodiments, the sensor data comprises intensity and duration of the pressure. In some embodiments, the smart case comprises a support for holding the smart night guard. In some embodiments, the smart case comprises a wireless charger for charging the smart night guard. In some embodiments, the wireless charger comprises a first induction coil, and the smart night guard comprises a second induction coil, wherein the smart night guard undergoes inductive charging when positioned so the first induction coil and the second induction coil are in proximity. In some embodiments, at least one of the smart case or the smart night guard comprises at least one magnet for positioning the smart night guard within the smart case to allow inductive charging. In some embodiments, the smart case comprises at least one magnet for positioning the smart night guard. In some embodiments, the smart case comprises a display for providing information to the user. In some embodiments, the smart case comprises a UV light source for disinfecting the smart night guard. In some embodiments, the smart case comprises an anti-microbial coating. In some embodiments, the output comprises an evaluation of the health condition of the user. In some embodiments, the health condition comprises an oral health condition or a sleeping disorder or condition. In some embodiments, the health condition comprises bruxism, sleep apnea, insomnia, snoring, restless leg syndrome, sleepwalking. In some embodiments, the output comprises a correlation between the at least one user activity and an intensity or severity of the health condition of the user. In some embodiments, the output comprises a relationship between the sensor data and the at least one behavioral activity. In some embodiments, the output comprises a risk prediction of a health condition of the user. In some embodiments, the risk prediction is generated by a machine learning classifier configured to assess the risk of the health condition based on the sensor data. In some embodiments, the risk prediction comprises a degree of risk of the health condition. In some embodiments, the output comprises a number of bruxism episodes, maximum and average force of each bruxism episode, maximum and average durations of the bruxism episodes, an amount or quality of sleep, compliance to wearing the device, or any combination thereof. In some embodiments, the output comprises a score generated based on number of bruxism episodes, maximum and average force of each bruxism episode, maximum and average durations of the bruxism episodes, an amount or quality of sleep, compliance to wearing the device, or any combination thereof. In some embodiments, the output comprises at least one of personal or community correlation between the health condition and behavioral activity or therapeutic treatment. In some embodiments, behavioral activity or therapeutic treatment comprises dietary activity, physical activity, personal well-being indicator, or any combination thereof. In some embodiments, dietary activity comprises consumption of caffeine, alcohol, fats, simple carbohydrates, complex carbohydrates, protein, vitamins, supplements, or any combination thereof. In some embodiments, physical activity comprises aerobic exercise, anaerobic exercise, stretching, meditation, yoga, or any combination thereof. In some embodiments, personal well-being indicator comprises self-reported stress level, energy level, mental state, or any combination thereof. In some embodiments, the electronic device is further configured to generate a recommendation based on the output. In some embodiments, the recommendation comprises modifying the at least one behavioral activity, beginning a new behavioral activity, or terminating the at least one behavioral activity. In some embodiments, the recommendation comprises at least one therapeutic treatment or activity for reducing at least one of frequency, force, of duration of bruxism episodes. In some embodiments, the recommendation comprises at least one therapeutic treatment or activity for improving sleep quality. In some embodiments, the recommendation comprises at least one therapeutic treatment or activity for improving a symptom of the health condition. In some embodiments, the electronic device is configured to determine user compliance to the recommendation. In some embodiments, user compliance is determined at least partly based on user input. In some embodiments, the system is configured to monitor the health condition over time by continuously or repeatedly collecting sensor data and user data comprising the at least one behavioral activity. In some embodiments, the system is configured to generate updated output over time. In some embodiments, the system is adapted for continuous, repeated, or periodic monitoring of the user for at least 1 week, 2 weeks, 3 weeks, 4 weeks, 5 weeks, 6 weeks, 7 weeks, 8 weeks, 9 weeks, 10 weeks, 11 weeks, or at least 12 weeks. In some embodiments, the smart night guard is not configured to provide a biofeedback mechanism for the health condition, the biofeedback mechanism comprising vibration, sounds, or electrical stimulation. In some embodiments, the at least one pressure sensor comprises a single circuit having an arch shape configured to allow detection of the pressure from any point of contact between upper and lower teeth. In some embodiments, the electronic device is configured integrate the sensor data and output with a third party application. In some embodiments, the electronic device is configured integrate the sensor data and output with data from a third party application or sensor. In some embodiments, the electronic device is configured to generate a report comprising historical sensor data, user data, and outputs. In some embodiments, the electronic device is configured to provide a user portal comprising historical sensor data, user data, outputs, smart night guard status, or any combination thereof.

In some aspects, disclosed herein is a smart night guard comprising: (a) an inner layer configured to detachably engage at least one tooth of a user; (b) at least one pressure sensor configured to generate sensor data comprising duration and intensity in response to detection of an application of pressure to the inner layer; and (c) a transmitter in communication with the at least one pressure sensor, said transmitter configured to transmit the sensor data obtained from the at least one pressure sensor. In some embodiments, the smart night guard further comprises at least one sensor. In some embodiments, the at least one sensor comprises a motion sensor, a temperature sensor, a pH sensor, a gyroscope, a geomagnetic sensor, or any combination thereof. In some embodiments, the sensor data comprises absolute orientation, angular velocity vectors, acceleration vectors, magnetic field strength vectors, linear acceleration vectors, gravity vectors, ambient temperature, or any combination thereof. In some embodiments, the smart night guard is configured to increase sampling rate of the at least one pressure sensor when the pressure exceeds a minimum threshold or a moving threshold. In some embodiments, the smart night guard comprises an anti-microbial coating. In some embodiments, the smart night guard is configured to transmit the sensor data when the pressure exceeds a minimum threshold or a moving threshold. In some embodiments, the smart night guard is configured to temporarily store the sensor data and transmit the sensor data when charging, upon receiving a signal from the smart night guard or electronic device, when the smart night guard is experiencing minimal motion, or any combination thereof. In some embodiments, the smart night guard is configured to detect improper device states indicative of a defect or malfunction. In some embodiments, at least one of the smart night guard, smart case, or electronic device provides a signal or message to discontinue use upon detection of the defect or malfunction. In some embodiments, the signal or message to discontinue use comprises a light, a sound, or written message or warning. In some embodiments, the smart night guard is configured to calibrate the sensor data based on pressure corresponding to maximum or minimum detected bite force. In some embodiments, the sensor data comprises intensity and duration of the pressure. In some embodiments, the smart case comprises a support for holding the smart night guard. In some embodiments, the smart case comprises a wireless charger for charging the smart night guard. In some embodiments, the wireless charger comprises a first induction coil, and the smart night guard comprises a second induction coil, wherein the smart night guard undergoes inductive charging when positioned so the first induction coil and the second induction coil are in proximity. In some embodiments, at least one of the smart case or the smart night guard comprises at least one magnet for positioning the smart night guard within the smart case to allow inductive charging. In some embodiments, the smart case comprises at least one magnet for positioning the smart night guard. In some embodiments, the smart case comprises a display for providing information to the user. In some embodiments, the smart case comprises a UV light source for disinfecting the smart night guard. In some embodiments, the smart case comprises an anti-microbial coating. In some embodiments, the output comprises an evaluation of the health condition of the user. In some embodiments, the health condition comprises an oral health condition or a sleeping disorder or condition. In some embodiments, the health condition comprises bruxism, sleep apnea, insomnia, snoring, restless leg syndrome, sleepwalking. In some embodiments, the output comprises a correlation between the at least one user activity and an intensity or severity of the health condition of the user. In some embodiments, the output comprises a relationship between the sensor data and the at least one behavioral activity. In some embodiments, the output comprises a risk prediction of a health condition of the user. In some embodiments, the risk prediction is generated by a machine learning classifier configured to assess the risk of the health condition based on the sensor data. In some embodiments, the risk prediction comprises a degree of risk of the health condition. In some embodiments, the output comprises a number of bruxism episodes, maximum and average force of each bruxism episode, maximum and average durations of the bruxism episodes, an amount or quality of sleep, compliance to wearing the device, or any combination thereof. In some embodiments, the output comprises a score generated based on number of bruxism episodes, maximum and average force of each bruxism episode, maximum and average durations of the bruxism episodes, an amount or quality of sleep, compliance to wearing the device, or any combination thereof. In some embodiments, the output comprises at least one of personal or community correlation between the health condition and behavioral activity or therapeutic treatment. In some embodiments, behavioral activity or therapeutic treatment comprises dietary activity, physical activity, personal well-being indicator, or any combination thereof. In some embodiments, dietary activity comprises consumption of caffeine, alcohol, fats, simple carbohydrates, complex carbohydrates, protein, vitamins, supplements, or any combination thereof. In some embodiments, physical activity comprises aerobic exercise, anaerobic exercise, stretching, meditation, yoga, or any combination thereof. In some embodiments, personal well-being indicator comprises self-reported stress level, energy level, mental state, or any combination thereof. In some embodiments, the electronic device is further configured to generate a recommendation based on the output. In some embodiments, the recommendation comprises modifying the at least one behavioral activity, beginning a new behavioral activity, or terminating the at least one behavioral activity. In some embodiments, the recommendation comprises at least one therapeutic treatment or activity for reducing at least one of frequency, force, of duration of bruxism episodes. In some embodiments, the recommendation comprises at least one therapeutic treatment or activity for improving sleep quality. In some embodiments, the recommendation comprises at least one therapeutic treatment or activity for improving a symptom of the health condition. In some embodiments, the electronic device is configured to determine user compliance to the recommendation. In some embodiments, user compliance is determined at least partly based on user input. In some embodiments, the system is configured to monitor the health condition over time by continuously or repeatedly collecting sensor data and user data comprising the at least one behavioral activity. In some embodiments, the system is configured to generate updated output over time. In some embodiments, the system is adapted for continuous, repeated, or periodic monitoring of the user for at least 1 week, 2 weeks, 3 weeks, 4 weeks, 5 weeks, 6 weeks, 7 weeks, 8 weeks, 9 weeks, 10 weeks, 11 weeks, or at least 12 weeks. In some embodiments, the smart night guard is not configured to provide a biofeedback mechanism for the health condition, the biofeedback mechanism comprising vibration, sounds, or electrical stimulation. In some embodiments, the at least one pressure sensor comprises a single circuit having an arch shape configured to allow detection of the pressure from any point of contact between upper and lower teeth. In some embodiments, the electronic device is configured integrate the sensor data and output with a third party application. In some embodiments, the electronic device is configured integrate the sensor data and output with data from a third party application or sensor. In some embodiments, the electronic device is configured to generate a report comprising historical sensor data, user data, and outputs. In some embodiments, the electronic device is configured to provide a user portal comprising historical sensor data, user data, outputs, smart night guard status, or any combination thereof.

In some aspects, disclosed herein is a smart case comprising: (a) a holder for receiving a smart night guard; (b) an inductive charger; (c) at least one magnet for positioning the smart night guard on the holder to enable inductive charging; and (d) a processor and non-transitory computer readable medium including instructions executable by the processor and configured to cause the processor to: (i) receive the sensor data transmitted by the smart night guard over a period of time; (ii) store the sensor data in a local memory; and (iii) send the sensor data to an electronic device. In some embodiments, the smart night guard further comprises at least one sensor. In some embodiments, the at least one sensor comprises a motion sensor, a temperature sensor, a pH sensor, a gyroscope, a geomagnetic sensor, or any combination thereof. In some embodiments, the sensor data comprises absolute orientation, angular velocity vectors, acceleration vectors, magnetic field strength vectors, linear acceleration vectors, gravity vectors, ambient temperature, or any combination thereof. In some embodiments, the smart night guard is configured to increase sampling rate of the at least one pressure sensor when the pressure exceeds a minimum threshold or a moving threshold. In some embodiments, the smart night guard comprises an anti-microbial coating. In some embodiments, the smart night guard is configured to transmit the sensor data when the pressure exceeds a minimum threshold or a moving threshold. In some embodiments, the smart night guard is configured to temporarily store the sensor data and transmit the sensor data when charging, upon receiving a signal from the smart night guard or electronic device, when the smart night guard is experiencing minimal motion, or any combination thereof. In some embodiments, the smart night guard is configured to detect improper device states indicative of a defect or malfunction. In some embodiments, at least one of the smart night guard, smart case, or electronic device provides a signal or message to discontinue use upon detection of the defect or malfunction. In some embodiments, the signal or message to discontinue use comprises a light, a sound, or written message or warning. In some embodiments, the smart night guard is configured to calibrate the sensor data based on pressure corresponding to maximum or minimum detected bite force. In some embodiments, the sensor data comprises intensity and duration of the pressure. In some embodiments, the smart case comprises a support for holding the smart night guard. In some embodiments, the smart case comprises a wireless charger for charging the smart night guard. In some embodiments, the wireless charger comprises a first induction coil, and the smart night guard comprises a second induction coil, wherein the smart night guard undergoes inductive charging when positioned so the first induction coil and the second induction coil are in proximity. In some embodiments, at least one of the smart case or the smart night guard comprises at least one magnet for positioning the smart night guard within the smart case to allow inductive charging. In some embodiments, the smart case comprises at least one magnet for positioning the smart night guard. In some embodiments, the smart case comprises a display for providing information to the user. In some embodiments, the smart case comprises a UV light source for disinfecting the smart night guard. In some embodiments, the smart case comprises an anti-microbial coating. In some embodiments, the output comprises an evaluation of the health condition of the user. In some embodiments, the health condition comprises an oral health condition or a sleeping disorder or condition. In some embodiments, the health condition comprises bruxism, sleep apnea, insomnia, snoring, restless leg syndrome, sleepwalking. In some embodiments, the output comprises a correlation between the at least one user activity and an intensity or severity of the health condition of the user. In some embodiments, the output comprises a relationship between the sensor data and the at least one behavioral activity. In some embodiments, the output comprises a risk prediction of a health condition of the user. In some embodiments, the risk prediction is generated by a machine learning classifier configured to assess the risk of the health condition based on the sensor data. In some embodiments, the risk prediction comprises a degree of risk of the health condition. In some embodiments, the output comprises a number of bruxism episodes, maximum and average force of each bruxism episode, maximum and average durations of the bruxism episodes, an amount or quality of sleep, compliance to wearing the device, or any combination thereof. In some embodiments, the output comprises a score generated based on number of bruxism episodes, maximum and average force of each bruxism episode, maximum and average durations of the bruxism episodes, an amount or quality of sleep, compliance to wearing the device, or any combination thereof. In some embodiments, the output comprises at least one of personal or community correlation between the health condition and behavioral activity or therapeutic treatment. In some embodiments, behavioral activity or therapeutic treatment comprises dietary activity, physical activity, personal well-being indicator, or any combination thereof. In some embodiments, dietary activity comprises consumption of caffeine, alcohol, fats, simple carbohydrates, complex carbohydrates, protein, vitamins, supplements, or any combination thereof. In some embodiments, physical activity comprises aerobic exercise, anaerobic exercise, stretching, meditation, yoga, or any combination thereof. In some embodiments, personal well-being indicator comprises self-reported stress level, energy level, mental state, or any combination thereof. In some embodiments, the electronic device is further configured to generate a recommendation based on the output. In some embodiments, the recommendation comprises modifying the at least one behavioral activity, beginning a new behavioral activity, or terminating the at least one behavioral activity. In some embodiments, the recommendation comprises at least one therapeutic treatment or activity for reducing at least one of frequency, force, of duration of bruxism episodes. In some embodiments, the recommendation comprises at least one therapeutic treatment or activity for improving sleep quality. In some embodiments, the recommendation comprises at least one therapeutic treatment or activity for improving a symptom of the health condition. In some embodiments, the electronic device is configured to determine user compliance to the recommendation. In some embodiments, user compliance is determined at least partly based on user input. In some embodiments, the system is configured to monitor the health condition over time by continuously or repeatedly collecting sensor data and user data comprising the at least one behavioral activity. In some embodiments, the system is configured to generate updated output over time. In some embodiments, the system is adapted for continuous, repeated, or periodic monitoring of the user for at least 1 week, 2 weeks, 3 weeks, 4 weeks, 5 weeks, 6 weeks, 7 weeks, 8 weeks, 9 weeks, 10 weeks, 11 weeks, or at least 12 weeks. In some embodiments, the smart night guard is not configured to provide a biofeedback mechanism for the health condition, the biofeedback mechanism comprising vibration, sounds, or electrical stimulation. In some embodiments, the at least one pressure sensor comprises a single circuit having an arch shape configured to allow detection of the pressure from any point of contact between upper and lower teeth. In some embodiments, the electronic device is configured integrate the sensor data and output with a third party application. In some embodiments, the electronic device is configured integrate the sensor data and output with data from a third party application or sensor. In some embodiments, the electronic device is configured to generate a report comprising historical sensor data, user data, and outputs. In some embodiments, the electronic device is configured to provide a user portal comprising historical sensor data, user data, outputs, smart night guard status, or any combination thereof.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a schematic diagram of a pressure sensor having an arch shape in a single circuit;

FIG. 2 shows one embodiment of a construction of two sensor layers in a pressure sensor;

FIG. 3 shows one embodiment of an electronics casing used in a smart night guard;

FIG. 4 shows one embodiment of an initial casing trimmed to size on cast teeth;

FIG. 5 shows one embodiment of the night guard after trimming of the second casing on cast teeth;

FIG. 6 shows one embodiment of data screens of a software application for monitoring bruxism, including analytics for the previous night and weekly trends;

FIG. 7 shows one embodiment of data screens of a software application for monitoring bruxism, including both personal and community data correlations;

FIG. 8 shows one embodiment of a data screen of a software application for monitoring bruxism, including a single score indicative of the level of bruxing for the previous night;

FIG. 9 shows a flow diagram illustrating a process for obtaining and uploading sensor data for analysis by the cloud;

FIG. 10 shows a flow diagram illustrating a process for obtaining and uploading sensor data for analysis by the electronic device (e.g., mobile device);

FIG. 11 shows a flow diagram illustrating a process for obtaining and uploading sensor data for analysis in which the data is stored by a smart case before it is provided to the electronic device and/or cloud for analysis; and

FIG. 12 shows an exemplary embodiment of an electronic device as described herein.

### DETAILED DESCRIPTION

Described herein are systems, apparatuses, software, and methods for monitoring and/or treating health conditions such as bruxism and/or associated symptoms such as poor sleep quality.

### Smart Night Guard

In some aspects, disclosed herein is a smart night guard configured to measure and record the bite pressure of the wearer. In some embodiments, the smart night guard is configured to be worn for long durations of time. In some embodiments, the smart night guard comprises one or more of the following components: sensors, a storage medium, a data transmitter, a battery, a battery charge controller, and a casing.

### Sensors

In some embodiments, the smart night guard comprises one or more sensors. In some embodiments, the smart night guard comprises at least one pressure sensor. In some embodiments, the pressure sensor is custom-fabricated with a unique arch shape to provide the ability to sense pressure from any point of upper and lower teeth contact. In some embodiments, the arch shape is unique in that it allows for pressure sensing around the entire dentition rather than in sparse, discrete sections and is wide enough to accommodate various sized dental arches. In some embodiments, the pressure sensor is constructed using a plurality of polymeric sheets impregnated with carbon black, or more broadly any conductive polymer, to form a certain thickness. In some embodiments, these sheets have the property of changing their resistivity in response to the application of external pressure. In some embodiments, two opposing conductors are positioned above and below the inner sheets, and cut to the same profile as the inner sheets but offset by a certain distance as to prevent direct contact of the conductors. In some embodiments, above and below each conductor is an additional carbon black material sheet cut at the same size as the original sheet. An example of this material stack-up is depicted in FIG. 2.

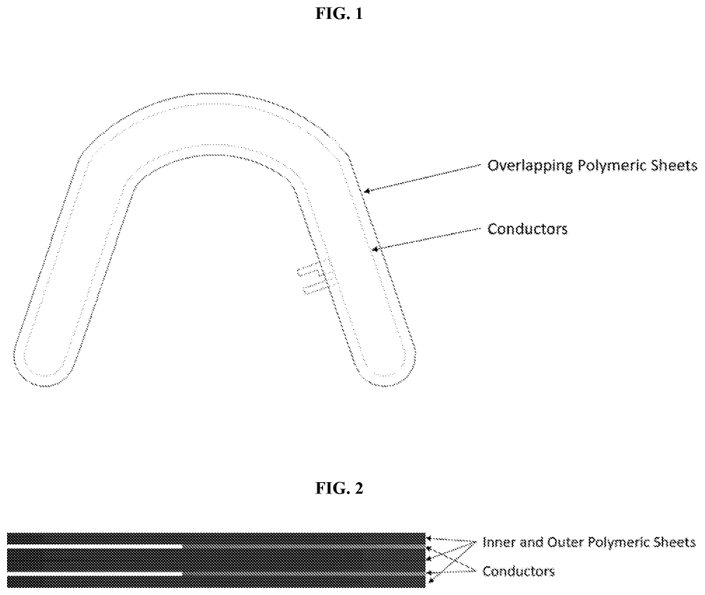

In some embodiments, the pressure sensor is assembled using the application of a heat sealer by applying both heat and pressure to the overlapping offset edges of carbon black material. This process bonds the edges of the layers of the sensor together with the conductors trapped inside. In one embodiment of the sensor, both conductors are composed of single, unsegmented pieces of copper (see FIG. 1). In another embodiment, one of the conductors is the same as previously described, however the other conductor is segmented into multiple distinct areas. This allows measurement of localized pressures at each segment on the sensor. In some embodiments, both conductors are segmented to localize both the application and reading of the pressure to each segment on the sensor. In some embodiments, each conductor segment is constructed with tabs for connection to the measurement apparatus. Alternative sensor materials, such as piezoelectrics, could replace the internal sensor layers while providing similar functionality. A motion and temperature sensor can also be included in the smart night guard. In some embodiments, a suitable motion and temperature sensor is included in the smart night guard. In some embodiments, the smart night guard comprises a gyroscope, accelerometer, and geomagnetic sensor. In some embodiments, the data gathered by the smart night guard allows output data on absolute orientation, angular velocity vectors, acceleration vectors, magnetic field strength vectors, linear acceleration vectors, gravity vectors, ambient temperature, or any combination thereof. For example, the data output by this device can be used in assessing the user's compliance to wearing the device, the user's movement while wearing the device, the user's respiratory rate, the user's heart rate, and the sleep state of the user.

### Recording Apparatus

In some aspects, the smart night guard comprises a recording apparatus or element. In some embodiments, the pressure sensor responds to changes in external pressure with a changed resistance as measured across the two conductors, or across each of the conductor segments. In some embodiments, a microcontroller is used to control both the application of voltage to one of the conductors of the pressure sensor and the measurement of voltage at the other conductor. In some embodiments, a voltage divider circuit is used to limit the maximum current applied to the sensor and to be able to measure the voltage drop across the sensor. A suitable microcontroller is a SAMD21E18A produced by Atmel and has an internal analog to digital converter (ADC). The microcontroller can constantly sample the device using a fixed rate (such as 10 Hertz), record the measured voltage value to a buffer, then save the buffered values to the memory device. A similar sampling action can be taken to collect the data from the motion and temperature sensor using the same microcontroller. In some embodiments, the microcontroller remains in a deep sleep state in which only the ADC is awake. In this embodiment, the device samples the pressure, motion, and temperature sensors at a fixed rate, but only saves the values to a buffer when any of value record fall outside of some predefined thresholds.

In another embodiment, instead of a predefined threshold, a moving threshold is used in which relative changes greater than a certain percentage of the past measurements trigger recording to the buffer. In some embodiments, regardless of the sampling method used, the microcontroller saves the buffer of sensor data to the memory storage device upon filling of the buffer. In some embodiments, the microcontroller is configured for monitoring the battery voltage level, for example, by using a voltage divider circuit. In some embodiments, the microcontroller is configured for communication with the data transmission module and can limit communications and control the data transmission module dependent on certain rules that allow automated data transmission while conserving energy usage. One rule is to only enable the data transmission module to communicate during charging of the device, as measured by monitoring of the battery. This would save power from the battery during other operations of the device and limit radiation from the data transmission module to certain periods. Another rule is to limit communications and only enable the data transmission module following a certain signal, such as an external key from the mobile application, to enable data transmission during special circumstances while the device is worn, such as during initial fitting at the dentist's office. Another rule is to monitor the motion sensor to limit data transmissions to states where the device is experiencing minimal motion, which would indicate it is not being worn by the user.

In some embodiments, the microcontroller is able to recognize improper device states. For example, an improper device state can include when the resistance across the sensor went to an improperly low value, indicating a short connection between the conductors, potentially from the presence of liquid that may indicate a breach in the body of the device. This information could be used to signal the user to discontinue use of the device.

In some embodiments, the microcontroller is configured to calibrate the biting force of the user. The microcontroller can recognize bite forces greater than had previously been recorded, store and retrieve the maximum bite force from the storage medium, and could calibrate past and future bite forces with respect to this maximum bite force. A similar feature could be completed for minimum bite forces.

### Storage Medium

The measurements that are gathered from the sensor can be saved to the device to an internal flash storage, for instance. An example of a suitable storage includes a 8-Mbit SPI Serial Flash Memory chip produced by Adesto Technologies (part number AT25DF081A).

### Data Transmitter

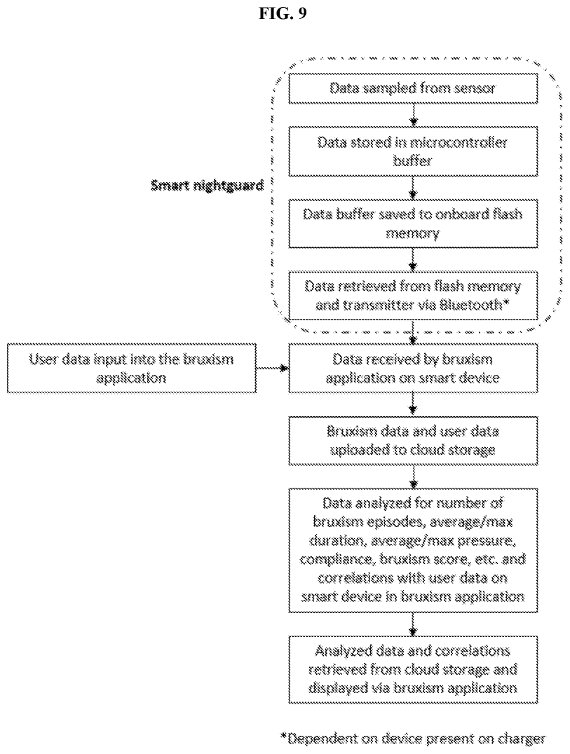

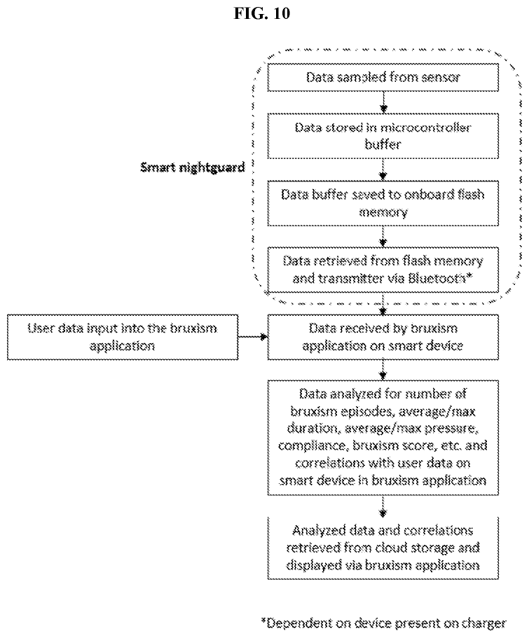

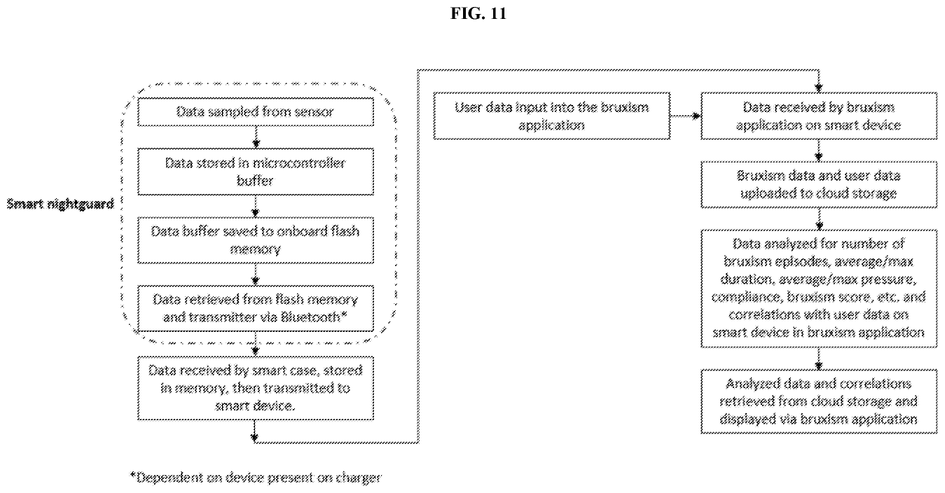

In some embodiments, the smart night guard comprises a wireless transmitter for sending stored data to another electronic device. In some embodiments, the wireless transmitter is a Bluetooth module. In some embodiments, the electronic device is a mobile device such as a smartphone, tablet, watch, fitness tracker, or personal computer. The data transmission process can be handled in packets of a predetermined size, for example, until all of the stored data on the storage media has been transferred. Options for the data flow are depicted in the data flow diagrams section. FIG. 9 shows a process for obtaining, transmitting, storing, and analyzing sensor data from the smart night guard in which the cloud performs the analysis. FIG. 10 shows a process for obtaining, transmitting, storing, and analyzing sensor data from the smart night guard in which the mobile device performs the analysis. FIG. 11 shows a process for obtaining, transmitting, storing, and analyzing sensor data from the smart night guard in which the smart case provides intermediate data storage before the sensor data is provided to the mobile device or cloud.

As an alternative to transmitting to a smart mobile device, the Bluetooth module, in some embodiments, communicates with a receiver module stored within a smart case. In some embodiments, the smart case provides charging of the device, such as by inductive charging, and stores the transmitted data using its own storage medium. In some embodiments, the data transmission commences automatically upon charging of the device, which saves power for the device in other states by not having to power the Bluetooth module.

Other means of data transmission include Wi-Fi, direct terminal connections such as USB, optical communications including light pulses or certain colors, audible transmissions, or tactile/vibratory transmission.

### Battery

In various embodiments the smart night guard comprises a battery. In some embodiments, the battery is an ultrathin lithium polymer battery (e.g., GMB201021). This battery is ideal due to its thin size and ability to be flexed as required to fit within the casing of the device.

### Battery Charge Controller

In some embodiments, the smart night guard comprises a battery charge controller. In some embodiments, the battery charge controller is maintained within an inductive charge receiver IC such as that produced by SemTech (part number TS51224). Alternatively, the circuit can incorporate a controller such as the DIO5158XM8 produced by DIOO. Both alternatives charge the battery cell at a constant current rate until a specific voltage (4.2V) is reached, at which point the charger switches to a constant-voltage mode. In addition to these embodiments, other controllers known to those of skill in the art are contemplated.

### Charger

In some embodiments, the smart night guard comprises circuitry for charging, such as for inductive charging, so that the electronics can be hermetically sealed and the device still recharged. In some embodiments, the wireless power receiver is the TS51224 produced by SemTech. In some embodiments, the device comprises a wireless power charging receiver coil, like that produced by Wurth Elektronik (part number 760308101208A). In some embodiments, the transmitter for the inductive charger is located within the smart case.

An alternative to inductive charging incorporates a plurality of exposed terminals on the device, which when connected to corresponding terminals on a charger, applies a constant-current/constant-voltage charge directly to the battery.

### Casing

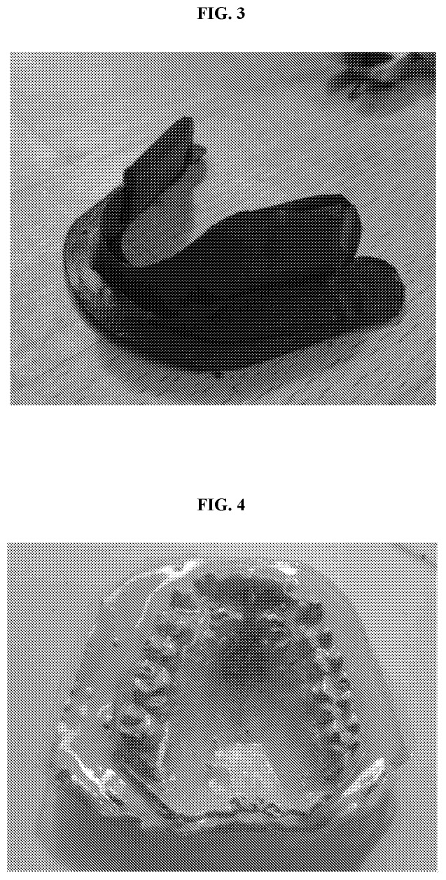

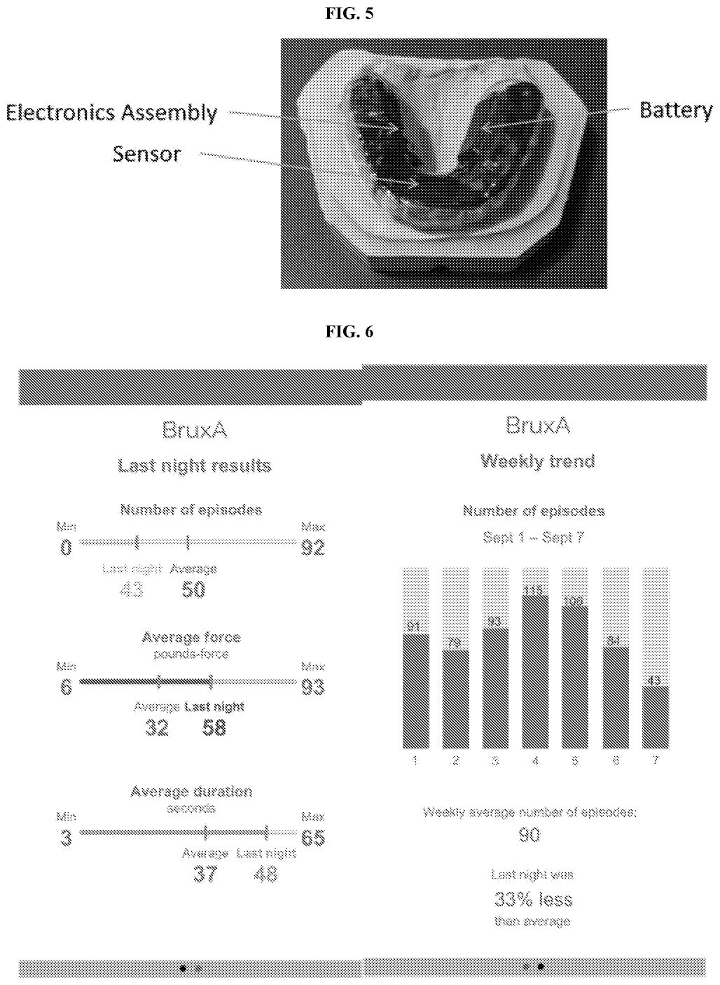

In some embodiments, the smart night guard comprises a casing. In some embodiments, the smart night guard comprises a first casing heat sealed packet around the sensor, electronics, and battery as shown in FIG. 3. In this embodiment, the casing is heat sealed around the entire perimeter such that fluids cannot reach the interior electronics. In some embodiments, the casing also positions the electronics and battery into two opposing wings, located on opposing parts of the roof of the mouth. In some embodiments, this initial casing of the electronics is then encased within two plastic (e.g., acrylic) sheets in the completed device. An exemplary method of construction of the device is as follows: first a single sheet of acrylic is vacuum thermoformed to the stone cast of the customer's upper teeth and roughly trimmed to size (shown in FIG. 4). In some embodiments, the initial casing is then placed on top of the formed sheet in such a manner so that the arch of the sensor aligns with the arch of the teeth cast. In some embodiments, the initial plastic sheet is heated simultaneously as a second plastic sheet is also heated for thermoforming. In some embodiments, the second plastic sheet is lowered onto the initial sheet and initial electronics casing and vacuum thermoformed. In some embodiments, the sheets are now bonded together and form a hermetic seal for the components within. While the device is cooling, a casting of the lower teeth can be used to imprint the device and provide a natural bite for the patient. In some embodiments, the entire device is trimmed so that the plastic doesn't extend beyond the gum line of the teeth on the distal side of the device and trimmed close to the casing on the proximal side of the device (as shown in FIG. 5).

Alternatives to the previously described methods for casing include other methods of manufacture currently available for custom night guards. In a lost-wax cast version, the electronics casing previously described is added to the device after manufacture and sealed over using a liquid acrylic. Alternatively, the wax model of the night guard includes a cavity where the electronics could be placed after casting and then sealed over using the same liquid acrylic method. The cavity can be created using a template of the same size as the electronics casing.

Another alternative embeds the electronics casing within the wax night guard model. When the wax is removed out of the cast, the electronics casing remains sealed in the cast acrylic. This method optionally can additionally employ supports or guides to ensure the electronics maintain the correct positioning during casting.

The casing is described with reference to creating a bruxism night guard for use on the upper teeth, however the same process could be used to create a device for the lower teeth, sleep apnea devices, sport mouth guards, and other dental appliances that would measure pressures or could be used to measure compliance of wearing the device.

### Smart Case

In some aspects, the system described herein comprises a smart case for use in monitoring and/or treating health conditions such as bruxism. In some embodiments, the smart case provides a number of functions as described throughout the present disclosure. In some embodiments, the smart case is configured to charge the battery of the smart night guard. In one embodiment, the night guard is inductively charged by the wireless transmission coils encased within the smart case. The smart case can be a plastic platform with a cavity that maintains the smart night guard in a specific location and orientation to ensure alignment of the transmission and reception coils of the smart case and smart night guard, respectively. In some instances, one or both of the smart case and smart night guard contain one or more magnet to promote close alignment of the two devices' inductive charging coils. In some embodiments, the smart case comprises a plurality of magnets. In some embodiments, the smart night guard comprises a plurality of magnets. In some embodiments, both the smart case and the smart night guard comprise a plurality of magnets. In some embodiments, the smart case and the smart night guard comprise one or more magnets and one or more corresponding metallic elements that match up with the one or more magnets. For example, the smart night guard may have a magnet and a metallic element positioned at the left and right sides, respectively, and the smart case may have a metallic element positioned to match with the magnet and a magnet positioned to match with the metallic element of the smart night guard. In some embodiments, the smart case comprises a plurality of metallic elements that are attracted to the plurality of magnets on the smart night guard. In some embodiments, the smart night guard comprises a plurality of metallic elements that are attracted to the plurality of magnets on the smart case.

In some embodiments, an inductive charging transmitter controller is located within the smart. In some embodiments, the controller uses, for example, the chips TS80002 and TS51231, both manufactured by Semtech, along with an inductive charging coil. In some embodiments, the chips are powered by a DC power supply that can in turn be supplied by a common household power outlet. In some embodiments, the smart case charging the smart night guard is used as a requirement to commence data transmission either to the smart case or to the bruxism application on the smart device. An alternative method of charging is for the smart case to contain a plurality of terminals that would correspond to the terminals of the smart night guard device and directly charge the battery of the smart night guard. In this case, a charger such as the DIO5158XM8 produced by DIOO can be enclosed within the smart case.

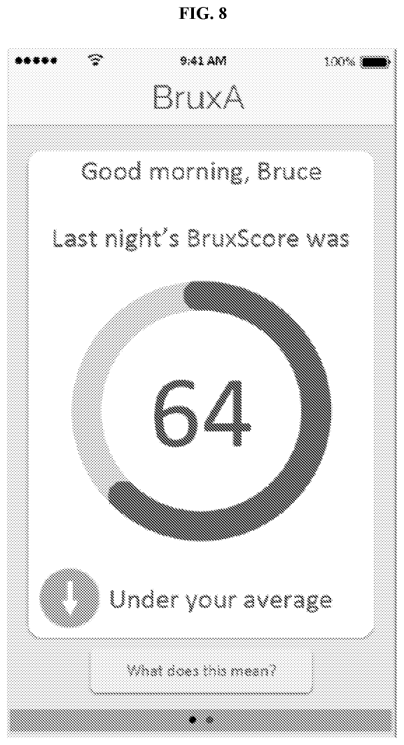

In some embodiments, the smart case provides a buffer for the sensor data collected from the smart night guard. In some embodiments, the smart case comprises a data reception chip, such as a Bluetooth or Wi-Fi module, that interacts directly with that of the smart night guard. The smart case can store this data, process this data for some type of output, or could output the data directly to another device, such as a computer, smartphone, or tablet. The processing of the data can analyze the sensor data to generate output comprising the number of bruxism episodes, the maximum and average force of each episode, the maximum and average durations of episodes, the amount or quality of sleep, compliance to wearing the device, or any combination thereof. In some embodiments, any of the above output can be optionally combined into a single score (an example of which is shown in FIG. 8). In some embodiments, any of this data is configured for display directly on a LCD display on the smart case. Alternately, in some embodiments, it can be conveyed using other visual means such a plurality of LED lights to indicate a scale or different ambient color lights to indicate a scale. Alternatively, the data can be conveyed using auditory means such as playing soothing sounds (e.g., environmental sounds like a babbling brook) to indicate a night of little bruxing, a thunderstorm to indicate heavy bruxing, or simple tones.

In some embodiments, the smart case comprises an ultraviolet light source to provide a means of sanitation for the smart night guard. In some embodiments, the smart case comprises holes in the night guard cavity and a sloping or angle to gravitationally divert any liquid on the night guard when it is placed into the smart case to a drainage location. In some embodiments, the smart case comprises a cover with a latching mechanism to facilitate transportation of the device.

### Bruxism Software Application

In some aspects, the system described herein comprises a health monitoring application such as, for example, a bruxism software application. In some embodiments, the application is configured to run on a smart device, such as a computer, tablet, smartphone, or wearable (e.g., a smart watch). In some embodiments, the application is configured to receive pressure, motion, temperature, battery status, time, or other data from the smart night guard or the smart case. The application can display this raw data directly to the user in numerical or graphical formats. In some embodiments, the application is configured to process the raw data to convert it into a format that is more meaningful for the user. This can include determining various analytics such as the number of bruxism episodes during a given time period (using predefined pressure and time thresholds or by analyzing the motion data for patterns recognizable as bruxism), the average and maximum pressure of bruxism episodes during a given time period, the average and maximum duration of bruxism episodes during a given time period, the amount, state, or quality of sleep, compliance to wearing the device, or any combination of this data can be combined into a single score to represent a user's bruxism on a scale, such as from 0-100. The application can store and track any of these analyses over time and display this back to the user in a numerical or graphical format over a given time period. In some embodiments, the application integrates with other smartphone applications such as Apple Health or Google Fit. For example, in some embodiments, the health monitoring application provides its sensor data and/or analytics to other applications. In some embodiments, the health monitoring application extracts data from other applications for integration into its analysis. In some embodiments, the user is able to view this presented data to determine their current level of a health condition such as bruxism with regards to their historical bruxism patterns and sleep quality. Examples of how this data can be displayed are shown in FIG. 6.

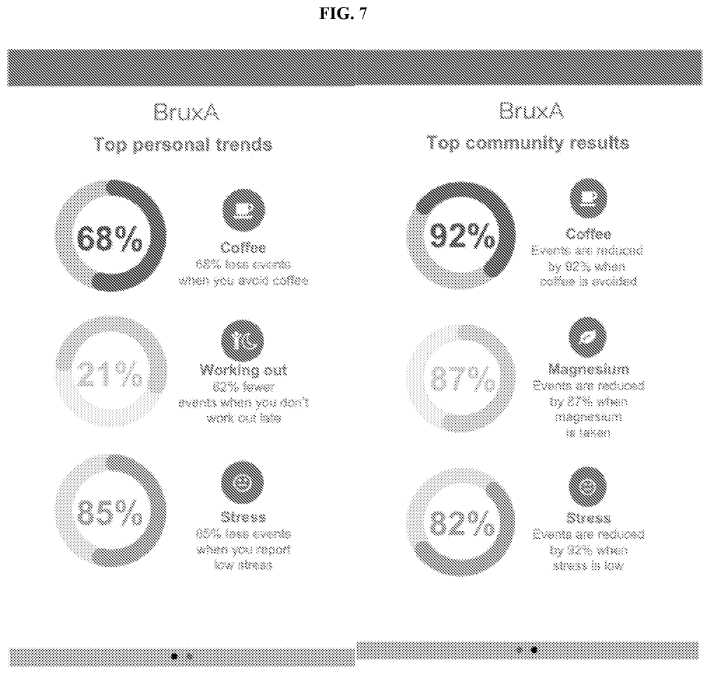

In some embodiments, the bruxism application is configured to accept input data from the user on a variety of preset or custom entered behavioral, emotional, psychological, medical or physiological inputs. For example, the user inputs within the application their current stress level on a nightly basis, whether they exercised during previous day, their consumption of alcohol, caffeine, or tobacco, or their consumption of different prescription or over-the-counter medicines. In some embodiments, the application then correlates these inputs with the data recorded by the smart night guard to present the user with activities that correlate with changes in their measured teeth clenching and grinding data or sleep quality. FIG. 7 shows an example of how this information could be presented to the user. An alternative to correlating this input data with the data from the smart night guard is to use machine learning to predict the level of bruxism for a given set of input activities.

In some embodiments, a web server aggregates and anonymizes the data from multiple users to analyze this data across multiple users. These “community” results can be displayed back to the smart night guard user within the bruxism application to help her determine what actions other users have found effective for reducing their bruxism. In some embodiments, the web server performs advanced analytics on the community results to generate more accurate models for generating predictions based on the community data. In some embodiments, the web server trains machine learning algorithms using the community results to generate prediction models. In some embodiments, the prediction models can be used to diagnosis or detect the presence of a health condition (e.g., bruxism, sleep apnea, etc.), identify an activity or therapy for treating or improving the health condition (e.g., reducing caffeine intake, taking melatonin supplements, etc.), and/or determine whether the health condition has improved, worsened, or remained unchanged. In some embodiments, the application is configured to evaluate user compliance (e.g., using accelerometer or geophysical location data to determine if the user is going to bed on time),

In some embodiments, the application is configured to provide storage of both the input data from the user as well as the data collected from the smart night guard. Alternately, the storage of this data, either in its raw or processed format, can be accomplished using an internet database, such as Firebase by Google. In this case, the processing of the data can be accomplished using a web application. In some embodiments, the application functions as a display means for the processed data retrieved from the online database. The processed or raw data can also be made available through a web or mobile application to other users authorized by the smart night guard user, such as a dentist, family, or friends. In some embodiments, the bruxism application is configured to use the data from the smart night guard to track user compliance with a dentist's recommendations for wearing the device. In some embodiments, the application is configured to send reminders or notifications to the user to encourage compliance with a dentist's recommendations for using the smart night guard.

In some embodiments, the application is configured to use bruxism data collected from the smart night guard to estimate the remaining life of the smart night guard, for example, before it is rebalanced to the user's occlusion or before it needs to be replaced. In some embodiments, an additional feature estimates the remaining life of the battery within the device based on the number of charge cycles of the device.

### Data Analysis

In some embodiments, the systems, methods, and media described herein use one or more algorithms analyzing sensor data and/or user data. In some embodiments, the algorithms utilize statistical modeling to generate predictions or estimates about the health condition and/or responsiveness to one or more behavioral activities or therapies. In some embodiments, machine learning algorithms are used for training prediction models and/or making predictions. In some embodiments, the algorithm predicts a likelihood or probability (e.g., probability of the presence of a health condition such as bruxism, or the probability of a particular treatment being effective for reducing severity or symptoms of bruxism). Various algorithms can be used to generate models that are used to make such predictions. In some instances, machine learning methods are applied to the generation of such models.

In some embodiments, a machine learning algorithm uses a supervised learning approach. In supervised learning, the algorithm generates a function from labeled training data. Each training example is a pair consisting of an input object and a desired output value. In some embodiments, an optimal scenario allows for the algorithm to correctly determine the class labels for unseen instances. In some embodiments, a supervised learning algorithm requires the user to determine one or more control parameters. These parameters are optionally adjusted by optimizing performance on a subset, called a validation set, of the training set. After parameter adjustment and learning, the performance of the resulting function is optionally measured on a test set that is separate from the training set. Regression methods are commonly used in supervised learning. Accordingly, supervised learning allows for a model or classifier to be generated or trained with training data in which the expected output is known in advance such as in calculating an adoption rate of a particular incentive offer type when historical adoption rates are known.

In some embodiments, a machine learning algorithm uses an unsupervised learning approach. In unsupervised learning, the algorithm generates a function to describe hidden structures from unlabeled data (e.g., a classification or categorization is not included in the observations). Since the examples given to the learner are unlabeled, there is no evaluation of the accuracy of the structure that is output by the relevant algorithm. Approaches to unsupervised learning include: clustering, anomaly detection, and neural networks.

In some embodiments, a machine learning algorithm learns in batches based on the training dataset and other inputs for that batch. In other embodiments, the machine learning algorithm performs on-line learning where the weights and error calculations are constantly updated. In some embodiments, the machine learning algorithm updates the prediction model based on new or updated user data (e.g., from a personalized user profile). For example, a machine learning algorithm can be applied to new or updated data to be re-trained or optimized to generate a new prediction model. In some embodiments, a machine learning algorithm or model is re-trained periodically as additional data becomes available (e.g., updated community data).

In some embodiments, the classifier or trained algorithm of the present disclosure comprises one feature space. In some cases, the classifier comprises two or more feature spaces. In some embodiments, the two or more feature spaces are distinct from one another. In some embodiments, the accuracy of the classification or prediction is improved by combining two or more feature spaces in a classifier instead of using a single feature space. The attributes generally make up the input features of the feature space and are labeled to indicate the classification of each case for the given set of input features corresponding to that case. For example, a user with severe bruxism whose sensor data indicates a significant reduction to light bruxism following reduction of caffeine consumption provides input features that can be used to train the model.

In some embodiments, an algorithm utilizes a predictive model such as a neural network, a decision tree, a support vector machine, or other applicable model. Using the training data, an algorithm is able to form a classifier for generating a classification or prediction according to relevant features. The features selected for classification can be classified using a variety of viable methods. In some embodiments, the trained algorithm comprises a machine learning algorithm. In some embodiments, the machine learning algorithm is selected from at least one of a supervised, semi-supervised and unsupervised learning, such as, for example, a support vector machine (SVM), a Naïve Bayes classification, a random forest, an artificial neural network, a decision tree, a K-means, learning vector quantization (LVQ), regression algorithm (e.g., linear, logistic, multivariate), association rule learning, deep learning, dimensionality reduction and ensemble selection algorithms. In some embodiments, the machine learning algorithm is a support vector machine (SVM), a Naïve Bayes classification, a random forest, or an artificial neural network. Machine learning techniques include bagging procedures, boosting procedures, random forest algorithms, and combinations thereof.

In some embodiments, a machine learning algorithm such as a classifier is tested using data that was not used for training to evaluate its predictive ability. In some embodiments, the predictive ability of the classifier is evaluated using one or more metrics. These metrics include accuracy, specificity, sensitivity, positive predictive value, negative predictive value, which are determined for a classifier by testing it against a set of independent cases. In some instances, an algorithm has an accuracy of at least about 75%, 80%, 85%, 90%, 95% or more, including increments therein, for at least about 50, 60, 70, 80, 90, 100, 110, 120, 130, 140, 150, 160, 170, 180, 190, or 200 independent cases, including increments therein. In some instances, an algorithm has a specificity of at least about 75%, 80%, 85%, 90%, 95% or more, including increments therein, for at least about 50, 60, 70, 80, 90, 100, 110, 120, 130, 140, 150, 160, 170, 180, 190, or 200 independent cases, including increments therein. In some instances, an algorithm has a sensitivity of at least about 75%, 80%, 85%, 90%, 95% or more, including increments therein, for at least about 50, 60, 70, 80, 90, 100, 110, 120, 130, 140, 150, 160, 170, 180, 190, or 200 independent cases, including increments therein. In some instances, an algorithm has a positive predictive value of at least about 75%, 80%, 85%, 90%, 95% or more, including increments therein, for at least about 50, 60, 70, 80, 90, 100, 110, 120, 130, 140, 150, 160, 170, 180, 190, or 200 independent cases, including increments therein. In some instances an algorithm has a negative predictive value of at least about 75%, 80%, 85%, 90%, 95% or more, including increments therein, for at least about 50, 60, 70, 80, 90, 100, 110, 120, 130, 140, 150, 160, 170, 180, 190, or 200 independent cases, including increments therein.

### Computing Systems and Software

In some embodiments, a system as described herein is configured to provide a software application such as a bruxism mobile application. In some embodiments, a system as described herein comprises a smart night guard comprising one or more sensors for determining physiological parameters such as bite pressure, temperature, motion or acceleration, or other sensory input. In some embodiments, a system as described herein comprises a network element for communicating with a server. In some embodiments, a system as described herein comprises a server. In some embodiments, the system is configured to upload to and/or download data from the server. In some embodiments, the server is configured to store sensor data, user input, output, and/or other information for the subject. In some embodiments, the server is configured to store historical data (e.g., past sensor data and/or user data) for the subject. In some embodiments, the server is configured to backup data from the system or apparatus.

In some embodiments, the system or apparatus is configured to encrypt data. In some embodiments, data on the server is encrypted. In some embodiments, the system or apparatus comprises a data storage unit or memory for storing data. In some embodiments, data encryption is carried out using Advanced Encryption Standard (AES). In some embodiments, data encryption is carried out using 128-bit, 192-bit, or 256-bit AES encryption. In some embodiments, data encryption comprises full-disk encryption of the data storage unit. In some embodiments, data encryption comprises virtual disk encryption. In some embodiments, data encryption comprises file encryption. In some embodiments, data that is transmitted or otherwise communicated between the system or apparatus and other devices or servers is encrypted during transit. In some embodiments, wireless communications between the system or apparatus and other devices or servers is encrypted. In some embodiments, data in transit is encrypted using a Secure Sockets Layer (SSL). In some embodiments, access to data stored on the system or apparatus as described herein requires user authentication. In some embodiments, access to data stored on the server as described herein requires user authentication.

An apparatus as described herein comprises a digital processing device that includes one or more hardware central processing units (CPUs) or general purpose graphics processing units (GPGPUs) that carry out the device's functions. The digital processing device further comprises an operating system configured to perform executable instructions. The digital processing device is optionally connected to a computer network. The digital processing device is optionally connected to the Internet such that it accesses the World Wide Web. The digital processing device is optionally connected to a cloud computing infrastructure. Suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein.

Typically, a digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing.

A digital processing device as described herein either includes or is operatively coupled to a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, a system or method as described herein generates a database as containing or comprising sensor data, user data, or other information. Some embodiments of the systems described herein are computer based systems. These embodiments include a CPU including a processor and memory which may be in the form of a non-transitory computer readable storage medium. These system embodiments further include software that is typically stored in memory (such as in the form of a non-transitory computer readable storage medium) where the software is configured to cause the processor to carry out a function. Software embodiments incorporated into the systems described herein contain one or more modules.

Some of the software embodiments described herein are configured to cause a processor to perform analysis comprising: receive sensor data over a period of time; obtain user data comprising at least one behavioral activity; and correlate the sensor data to the at least one behavioral activity to generate an output indicative of the relationship between a health condition such as bruxism with the at least one behavioral activity. For example, the analysis may determine that the duration and/or intensity of teeth grinding is reduced on nights when the subject engages in moderate exercise for at least 30 minutes. In some instances, the analysis incorporates additional sensor data aside from data from the smart night guard such as, for example, Fitbit data which can be used to infer physical activity.

In various embodiments, an apparatus comprises a computing device or component such as a digital processing device. In some of the embodiments described herein, a digital processing device includes a display to send visual information to a user. Non-limiting examples of displays suitable for use with the systems and methods described herein include a liquid crystal display (LCD), a thin film transistor liquid crystal display (TFT-LCD), an organic light emitting diode (OLED) display, an OLED display, an active-matrix OLED (AMOLED) display, or a plasma display.

A digital processing device, in some of the embodiments described herein includes an input device to receive information from a user. Non-limiting examples of input devices suitable for use with the systems and methods described herein include a keyboard, a mouse, trackball, track pad, or stylus. In some embodiments, the input device is a touch screen or a multi-touch screen.

The systems and methods described herein typically include one or more non-transitory computer readable storage media encoded with a program including instructions executable by the operating system of an optionally networked digital processing device. In some embodiments of the systems and methods described herein, the non-transitory storage medium is a component of a digital processing device that is a component of a system or is utilized in a method. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

Typically the systems and methods described herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages. The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

Typically, the systems and methods described herein include and/or utilize one or more databases. In view of the disclosure provided herein, those of skill in the art will recognize that many databases are suitable for storage and retrieval of baseline datasets, files, file systems, objects, systems of objects, as well as data structures and other types of information described herein. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

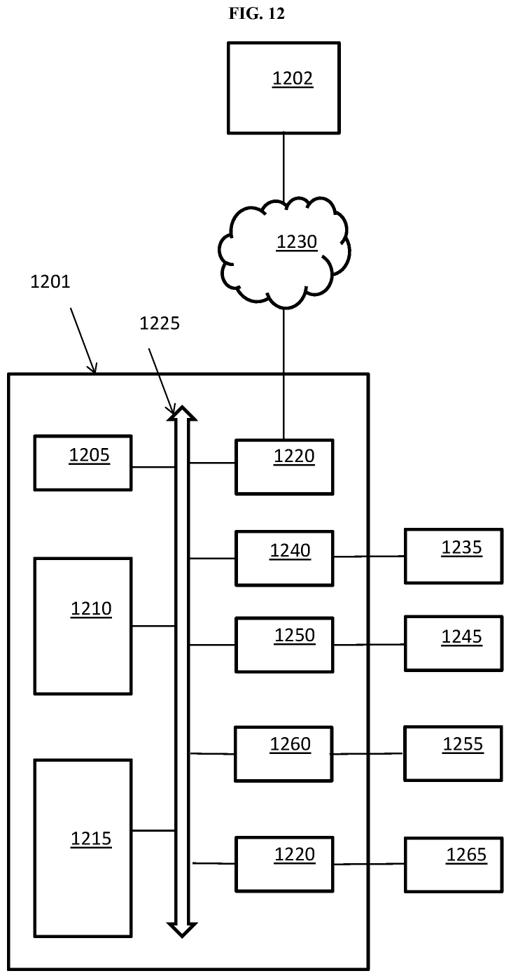

FIG. 12 shows an exemplary embodiment of a system as described herein comprising an apparatus such as a digital processing device 1201. The digital processing device 1201 includes a software application configured to monitor or treat a health condition such as bruxism. The digital processing device 1201 may include a central processing unit (CPU, also “processor” and “computer processor” herein) 1205, which can be a single core or multi-core processor, or a plurality of processors for parallel processing. The digital processing device 1201 also includes either memory or a memory location 1210 (e.g., random-access memory, read-only memory, flash memory), electronic storage unit 1215 (e.g., hard disk), communication interface 1220 (e.g., network adapter, network interface) for communicating with one or more other systems, and peripheral devices, such as cache. The peripheral devices can include storage device(s) or storage medium 1265 which communicate with the rest of the device via a storage interface 1270. The memory 1210, storage unit 1215, interface 1220 and peripheral devices are configured to communicate with the CPU 1205 through a communication bus 1225, such as a motherboard. The digital processing device 1201 can be operatively coupled to a computer network (“network”) 1230 with the aid of the communication interface 1220. The network 1230 can comprise the Internet. The network 1230 can be a telecommunication and/or data network.

The digital processing device 1201 includes input device(s) 1245 to receive information from a user, the input device(s) in communication with other elements of the device via an input interface 1250. The digital processing device 1201 can include output device(s) 1255 that communicates to other elements of the device via an output interface 1260.

The CPU 1205 is configured to execute machine-readable instructions embodied in a software application or module. The instructions may be stored in a memory location, such as the memory 1210. The memory 1210 may include various components (e.g., machine readable media) including, but not limited to, a random access memory component (e.g., RAM) (e.g., a static RAM “SRAM”, a dynamic RAM “DRAM, etc.), or a read-only component (e.g., ROM). The memory 1210 can also include a basic input/output system (BIOS), including basic routines that help to transfer information between elements within the digital processing device, such as during device start-up, may be stored in the memory 1210.

The storage unit 1215 can be configured to store files, such as health or risk parameter data, e.g., individual health or risk parameter values, health or risk parameter value maps, and value groups. The storage unit 1215 can also be used to store operating system, application programs, and the like. Optionally, storage unit 1215 may be removably interfaced with the digital processing device (e.g., via an external port connector (not shown)) and/or via a storage unit interface. Software may reside, completely or partially, within a computer-readable storage medium within or outside of the storage unit 1215. In another example, software may reside, completely or partially, within processor(s) 1205.

Information and data can be displayed to a user through a display 1235. The display is connected to the bus 1225 via an interface 1240, and transport of data between the display other elements of the device 1201 can be controlled via the interface 1240.

Methods as described herein can be implemented by way of machine (e.g., computer processor) executable code stored on an electronic storage location of the digital processing device 1201, such as, for example, on the memory 1210 or electronic storage unit 1215. The machine executable or machine readable code can be provided in the form of a software application or software module. During use, the code can be executed by the processor 1205. In some cases, the code can be retrieved from the storage unit 1215 and stored on the memory 1210 for ready access by the processor 1205. In some situations, the electronic storage unit 1215 can be precluded, and machine-executable instructions are stored on memory 1210.

In some embodiments, a remote device 1202 is configured to communicate with the digital processing device 1201, and may comprise any mobile computing device, non-limiting examples of which include a tablet computer, laptop computer, smartphone, or smartwatch. For example, in some embodiments, the remote device 1202 is a smartphone of the user that is configured to receive information from the digital processing device 1201 of the apparatus or system described herein in which the information can include a summary, sensor data, user data, output, or other data. In some embodiments, the remote device 1202 is a server on the network configured to send and/or receive data from the apparatus or system described herein.

Some embodiments of the systems and methods described herein are configured to generate a database containing or comprising of one or more sensor values. A database, as described herein, is configured to function as, for example, a lookup table for healthcare providers, other medical industry professionals and/or other end users. In these embodiments of the systems and methods described herein, sensor values are presented in a database so that a user is able to, for example, identify whether a parameter of a specific subject falls within or outside of a threshold value. In some embodiments, the database is stored on a server on the network. In some embodiments the database is stored locally on the apparatus (e.g., the monitor component of the apparatus). In some embodiments, the database is stored locally with data backup provided by a server.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A system for monitoring a health condition, comprising:
(a) a smart night guard comprising:
(i) an inner layer configured to detachably engage at least one tooth of a user;
(ii) at least one pressure sensor configured to generate sensor data in response to detection of an application of pressure to the inner layer; and
(iii) a transmitter in communication with the at least one pressure sensor, said transmitter configured to transmit the sensor data obtained from the at least one pressure sensor;
(b) a smart case comprising a processor and non-transitory computer readable medium including instructions executable by the processor and configured to cause the processor to:
(i) receive the sensor data transmitted by the smart night guard over a period of time;
(ii) store the sensor data in a local memory; and
(iii) send the sensor data to an electronic device;
(c) the electronic device comprising a processor and non-transitory computer readable medium including instructions executable by the processor and configured to cause the processor to:
(i) receive the sensor data transmitted by the smart case over a time period;
(ii) obtain user data comprising at least one user activity that is temporally proximate to the time period; and
(iii) analyze the sensor data and the user data, thereby generating an output indicative of the health condition.

2. The system of claim 1, wherein the smart night guard further comprises at least one sensor.

3. The system of claim 2, wherein the at least one sensor comprises a motion sensor, a temperature sensor, a pH sensor, a gyroscope, a geomagnetic sensor, or any combination thereof.

4. The system of claim 3, wherein the sensor data comprises absolute orientation, angular velocity vectors, acceleration vectors, magnetic field strength vectors, linear acceleration vectors, gravity vectors, ambient temperature, or any combination thereof.

5. The system of claim 1, wherein the smart night guard is configured to increase sampling rate of the at least one pressure sensor when the pressure exceeds a minimum threshold or a moving threshold.

6. The system of claim 1, wherein the smart night guard comprises an anti-microbial coating.

7. The system of claim 1, wherein the smart night guard is configured to transmit the sensor data when the pressure exceeds a minimum threshold or a moving threshold.

8. The system of claim 1, wherein the smart night guard is configured to temporarily store the sensor data and transmit the sensor data when charging, upon receiving a signal from the smart night guard or electronic device, when the smart night guard is experiencing minimal motion, or any combination thereof.

9. The system of claim 1, wherein the smart night guard is configured to detect improper device states indicative of a defect or malfunction.

10. The system of claim 9, wherein at least one of the smart night guard, smart case, or electronic device provides a signal or message to discontinue use upon detection of the defect or malfunction.

11. The system of claim 10, wherein the signal or message to discontinue use comprises a light, a sound, or written message or warning.

12. The system of claim 1, wherein the smart night guard is configured to calibrate the sensor data based on pressure corresponding to maximum or minimum detected bite force.

13. The system of claim 1, wherein the sensor data comprises intensity and duration of the pressure.

14. The system of claim 1, wherein the smart case comprises a support for holding the smart night guard.

15. The system of claim 1, wherein the smart case comprises a wireless charger for charging the smart night guard.

16. The system of claim 15, wherein the wireless charger comprises a first induction coil, and the smart night guard comprises a second induction coil, wherein the smart night guard undergoes inductive charging when positioned so the first induction coil and the second induction coil are in proximity.

17. The system of claim 16, wherein at least one of the smart case or the smart night guard comprises at least one magnet for positioning the smart night guard within the smart case to allow inductive charging.

18. The system of claim 1, wherein the smart case comprises at least one magnet for positioning the smart night guard.

19. The system of claim 1, wherein the smart case comprises a display for providing information to the user.

20. The system of claim 1, wherein the smart case comprises a UV light source for disinfecting the smart night guard.

21. The system of claim 1, wherein the smart case comprises an anti-microbial coating.

22. The system of claim 1, wherein the output comprises an evaluation of the health condition of the user.

23. The system of claim 22, wherein the health condition comprises an oral health condition or a sleeping disorder or condition.

24. The system of claim 22, wherein the health condition comprises bruxism, sleep apnea, insomnia, snoring, restless leg syndrome, sleepwalking.

25. The system of claim 24, wherein the output comprises a correlation between the at least one user activity and an intensity or severity of the health condition of the user.

26. The system of claim 1, wherein the output comprises a relationship between the sensor data and the at least one behavioral activity.

27. The system of claim 1, wherein the output comprises a risk prediction of a health condition of the user.

28. The system of claim 27, wherein the risk prediction is generated by a machine learning classifier configured to assess the risk of the health condition based on the sensor data.

29. The system of claim 27, wherein the risk prediction comprises a degree of risk of the health condition.

30. The system of claim 1, wherein the output comprises a number of bruxism episodes, maximum and average force of each bruxism episode, maximum and average durations of the bruxism episodes, an amount or quality of sleep, compliance to wearing the device, or any combination thereof.

31. The system of claim 1, wherein the output comprises a score generated based on number of bruxism episodes, maximum and average force of each bruxism episode, maximum and average durations of the bruxism episodes, an amount or quality of sleep, compliance to wearing the device, or any combination thereof.

32. The system of claim 1, wherein the output comprises at least one of personal or community correlation between the health condition and behavioral activity or therapeutic treatment.

33. The system of claim 32, wherein behavioral activity or therapeutic treatment comprises dietary activity, physical activity, personal well-being indicator, or any combination thereof.

34. The system of claim 33, wherein dietary activity comprises consumption of caffeine, alcohol, fats, simple carbohydrates, complex carbohydrates, protein, vitamins, supplements, or any combination thereof.

35. The system of claim 33, wherein physical activity comprises aerobic exercise, anaerobic exercise, stretching, meditation, yoga, or any combination thereof.

36. The system of claim 33, wherein personal well-being indicator comprises self-reported stress level, energy level, mental state, or any combination thereof.

37. The system of claim 1, wherein the electronic device is further configured to generate a recommendation based on the output.

38. The system of claim 37, wherein the recommendation comprises modifying the at least one behavioral activity, beginning a new behavioral activity, or terminating the at least one behavioral activity.

39. The system of claim 37, wherein the recommendation comprises at least one therapeutic treatment or activity for reducing at least one of frequency, force, of duration of bruxism episodes.

40. The system of claim 37, wherein the recommendation comprises at least one therapeutic treatment or activity for improving sleep quality.

41. The system of claim 37, wherein the recommendation comprises at least one therapeutic treatment or activity for improving a symptom of the health condition.

42. The system of claim 37, wherein the electronic device is configured to determine user compliance to the recommendation.

43. The system of claim 42, wherein user compliance is determined at least partly based on user input.

44. The system of claim 1, wherein the system is configured to monitor the health condition over time by continuously or repeatedly collecting sensor data and user data comprising the at least one behavioral activity.

45. The system of claim 44, wherein the system is configured to generate updated output over time.

46. The system of claim 1, wherein the system is adapted for continuous, repeated, or periodic monitoring of the user for at least 1 week, 2 weeks, 3 weeks, 4 weeks, 5 weeks, 6 weeks, 7 weeks, 8 weeks, 9 weeks, 10 weeks, 11 weeks, or at least 12 weeks.

47. The system of claim 1, wherein the smart night guard is not configured to provide a biofeedback mechanism for the health condition, the biofeedback mechanism comprising vibration, sounds, or electrical stimulation.

48. The system of claim 1, wherein the at least one pressure sensor comprises a single circuit having an arch shape configured to allow detection of the pressure from any point of contact between upper and lower teeth.

49. The system of claim 1, wherein the electronic device is configured integrate the sensor data and output with a third party application.

50. The system of claim 1, wherein the electronic device is configured integrate the sensor data and output with data from a third party application or sensor.

51. The system of claim 1, wherein the electronic device is configured to generate a report comprising historical sensor data, user data, and outputs.

52. The system of claim 1, wherein the electronic device is configured to provide a user portal comprising historical sensor data, user data, outputs, smart night guard status, or any combination thereof.

53. A system for monitoring a health condition, comprising:
(a) a smart night guard comprising:
(i) an inner layer configured to detachably engage at least one tooth of a user;
(ii) at least one pressure sensor configured to generate sensor data in response to detection of an application of pressure to the inner layer; and
(iii) a transmitter in communication with the at least one pressure sensor, said transmitter configured to transmit the sensor data obtained from the at least one pressure sensor;
(b) a smart case comprising a processor and non-transitory computer readable medium including instructions executable by the processor and configured to cause the processor to:
(i) receive the sensor data transmitted by the smart night guard over a period of time;
(ii) store the sensor data in a local memory; and
(iii) send the sensor data to an electronic device.

54. A system for monitoring a health condition, comprising:
(a) a smart night guard comprising:
(i) an inner layer configured to detachably engage at least one tooth of a user;
(ii) at least one pressure sensor configured to generate sensor data in response to detection of an application of pressure to the inner layer; and
(iii) a transmitter in communication with the at least one pressure sensor, said transmitter configured to transmit the sensor data obtained from the at least one pressure sensor;
(b) the electronic device comprising a processor and non-transitory computer readable medium including instructions executable by the processor and configured to cause the processor to:
(i) receive the sensor data transmitted by the smart night guard over a time period;
(ii) obtain user data comprising at least one user activity that is temporally proximate to the time period; and
(iii) analyze the sensor data and the user data, thereby generating an output indicative of the health condition.

55. A smart night guard comprising:
(a) an inner layer configured to detachably engage at least one tooth of a user;
(b) at least one pressure sensor configured to generate sensor data comprising duration and intensity in response to detection of an application of pressure to the inner layer; and
(c) a transmitter in communication with the at least one pressure sensor, said transmitter configured to transmit the sensor data obtained from the at least one pressure sensor.

56. A smart case comprising:
(a) a holder for receiving a smart night guard;
(b) an inductive charger;
(c) at least one magnet for positioning the smart night guard on the holder to enable inductive charging; and
(d) a processor and non-transitory computer readable medium including instructions executable by the processor and configured to cause the processor to:
(i) receive the sensor data transmitted by the smart night guard over a period of time;
(ii) store the sensor data in a local memory; and
(iii) send the sensor data to an electronic device.
